(19) United States
(12) Patent Application Publication
(10) Pub. No.: US 2023/0178194 A1
(43) Pub. Date: Jun. 8, 2023

(54) DOCUMENT INFORMATION SHARING SYSTEM BETWEEN ORGANIZATIONS

(71) Applicant: IRYOU JYOUHOU GIJYUTU KENKYUSHO CORPORATION, Yame-gun Fukuoka (JP)

(72) Inventor: Shinkichi HIMENO, Yame-gun Fukuoka (JP)

(73) Assignee: IRYOU JYOUHOU GIJYUTU KENKYUSHO CORPORATION, Yame-gun Fukuoka (JP)

(21) Appl. No.: 18/011,022

(22) PCT Filed: Nov. 3, 2021
(86) PCT No.: PCT/JP2021/040470

§ 371 Date: Dec. 16, 2022

(30) Foreign Application Priority Data
Nov. 17, 2020 (JP) 2020-190588

(51) Int. Cl. G16H 10/60; G06F 16/25

(52) U.S. Cl. CPC G16H 10/60; G06F 16/258; H04L 67/06

(57) ABSTRACT

An inter-organization document information sharing system for sharing document information between electronic health records of multiple organizations between which configurations of document categories or description items in the document categories do not necessarily match each other includes transmission/reception format definition configured to define a versatile transmission/reception format, transmission/reception format entry/transmission configured to extract information from description items in document categories of transmission-side electronic health records, to enter the information into the transmission/reception format, and to transmit the transmission/reception format, and transmission/reception format reception/recording configured to receive the transmission/reception format and to record the information entered into the transmission/reception format in corresponding description items in document categories of reception-side electronic health records.

### TECHNICAL FIELD

The present invention relates to a system that allows multiple organizations, in particular, medical institutions and/or nursing homes to share documents created by them.

### BACKGROUND ART

At medical, nursing care, and other sites, staff members in multiple job categories such as doctor, nurse, and care worker create, as electronic health records, documents such as basic information documents at the entry into a hospital or nursing home, medical or care plan documents, execution plan documents, execution result report documents, and various certificates provided to users. Information is shared between the staff members or different job categories through these documents.

When a patient or user uses a different medical institution or nursing home, the different medical institution or nursing home needs to acquire an enormous amount of information. Reacquiring the information from the beginning requires much time and effort. Also, the patient or user feels painful when they is asked about the same things a number of times. Particularly when it is necessary to quickly check information in case of emergency or the like, it is necessary to quickly electronically refer to the electronic health records of the medical institution or nursing home that the patient or user previously used.

Transmitting documents accumulated in a server to a server in another facility is never easy. First, the transmission-side staff and the reception-side staff previously confer with each other about the internal structure or specification of documents that the transmission side wants to transmit, the transmission procedure, the reception procedure, and the like and then transmit or receive the documents. For this reason, it is difficult to transmit or receive the documents flexibly and responsively.

Web services have been used since around 2000. A procedure document called Web Services Description Language (WSDL) describing the internal structure or specification of a document to be transmitted, the transmission procedure, the reception procedure, and the like is previously published on the transmission-side server. The reception-side server first reads the WSDL document and automatically creates a reception program (broker program). The reception-side server then transmits a document transmission request to the transmission-side server and receives and captures the transmitted document. This method eliminates the need for the staff to previously confer with each other and thus allows the staff to transmit or receive the document flexibly and responsively. This method allows for, for example, simultaneous display of information sources such as weather, news, stock prices, and the like from multiple servers and thus is a current standard method for performing website creation or the like.

With the progress of population aging, multiple medical institutions, nursing homes, and the like have been required to treat or support individual elderly persons comprehensively in cooperation with each other in order to realize community medicine or care.

To meet this requirement, the medical institutions and/or nursing homes need to make information such as electronic health records being created by them available to each other. Electronic health records consist of various document categories such as documents created by doctors, nurses, and care workers. Those document categories include documents describing information generally collected at the time point of entry into the facility or the like, documents on examination, treatment, or intervention plans based on the information, post-intervention report documents, and the like.

In each document category, there is at least one information item to be described (hereafter referred to as a description item). Products such as ID-Link (a registered trademark of NEC Corporation) and HumanBridge (a registered trademark of Fujitsu Ltd.) are often used.

When exchanging documents such as electronic health records using any of those products, the supply side creates documents (SSMIX-II) in which description items in document categories to be disclosed are converted into a standardized format and transmits the documents using the above-mentioned Web services.

Background art literature relating to the present application includes the following.

### CITATION LIST

### Patent Literature

[Patent Literature 1] Japanese Unexamined Patent Application Publication No. 2002-117142

### Non-Patent Literature

[Non-Patent Literature 1] https://jpn.nec.com/medical_ healthcare/solution/id-link/index.html

### SUMMARY OF INVENTION

### Technical Problem

A medical institution or nursing home is able to simply view the contents of the electronic health records of another medical institution or nursing home through a Web browser as long as the supply side attaches the above-mentioned WSDL to its electronic health records and discloses them to the view side.

However, the document categories of electronic health records or the layout of description items vary among medical institutions and nursing homes. It is not easy to find out where the target information is recorded, and it is often difficult to quickly reach the document in which the information is recorded.

When the reception-side facility attempts to capture found description items into its electronic health records, it has to copy and paste each description item manually. This means that it is difficult to acquire a great amount of required and systemic information.

With respect to SSMIX-II, the supply side creates disclosure documents by converting document categories to be disclosed among its electronic health records into formats defined in SSMIX-II.

Since document categories and the layout of description items vary among medical institution and nursing homes, the supply side has to create an SSMIX-II document creation program for disclosure for each of document categories to be disclosed among its electronic health records.

The workload of at least more than three man-days and the cost of several hundred thousand yen are required for each document category.

Also, the document categories of electronic health records and the layout of description items often vary between the reception side and the supply side. For this reason, when the reception-side facility attempts to capture description items into its electronic health records, it has to copy and paste each description item manually. This means that it is difficult to acquire a great amount of required and systemic information, as in disclosure using the above-mentioned Web services.

The reception-side facility may automatically capture description items into its electronic health records. However, the document categories of the electronic health records and the layout of description items of the supply-side facility differ from those of the reception-side facility. For this reason, it is necessary to create a non-versatile description item capture program for each of the electronic health records of the supply-side medical institution or nursing home, resulting in high cost.

Actually, the Saga University constructed a 300-million yen-budget system for causing university hospitals to previously accumulate electronic health records and to continue treatment using the accumulated electronic health record information in the event of a disaster. However, the universities had to input accumulation information one by one manually due to a lack of the compatibility between their electronic health records. In the end, this system was left to stand with no information accumulated. Unfortunately, this system was pointed out as an inappropriate spending by the Board of Audit of Japan.

The present invention has been made to solve the above problems with the background art, and an object thereof is to provide a versatile inter-organization document information sharing system that allows even medical institutions and/or nursing homes having electronic health records different from each other in document category and description item layout to transmit or receive information versatilely without having to cope with the individual differences, as well as allows the reception side to automatically capture information in accordance with document categories and description item layout specific to its electronic health records.

### Solution to Problem

As means for accomplishing the above object, an inter-organization document information sharing system according to claim 1 is an inter-organization document information sharing system for sharing document information between electronic health records of multiple organizations between which configurations of document categories or description items in the document categories do not necessarily match each other. The inter-organization document information sharing system includes transmission/reception format definition means configured to define a versatile transmission/reception format, transmission/reception format entry/transmission means configured to extract information from description items in document categories of transmission-side electronic health records, to enter the information into the transmission/reception format, and to transmit the transmission/reception format, and transmission/reception format reception/recording means configured to receive the transmission/reception format and to record the information entered into the transmission/reception format in corresponding description items in document categories of reception-side electronic health records.

According to an inter-organization document information sharing system of claim 2, in the inter-organization document information sharing system of claim 1, the transmission/reception format definition means defines a list of tags indicating description items that need to be shared.

According to an inter-organization document information sharing system of claim 3, in the inter-organization document information sharing system of claim 1 or 2, the transmission/reception format entry/transmission means includes transmission-side map definition means configured to define correspondences between the list of tags indicating the description items that need to be shared and the corresponding description items in the document categories of the transmission-side electronic health records and transmission description item entry means configured to enter information in the corresponding description items in the document categories into the list of tags indicating the description items that need to be shared on the basis of the transmission-side map definition means.

In an inter-organization document information sharing system of claim 4, in the inter-organization document information sharing system of any one of claims 1 to 3, the transmission/reception format reception/recording means includes reception-side map definition means configured to define correspondences between the list of tags indicating the description items that need to be shared and the corresponding description items in the document categories of the reception-side electronic health records and reception description item recording means configured to record the information entered into the list of tags indicating the description items that need to be shared in the corresponding description items in the document categories of the reception-side electronic health records on the basis of the reception-side map definition means.

According to an inter-organization document information sharing system of claim 5, the inter-organization document information sharing system of any one of claims 1 to 4 includes description item multiple entry means configured to allow multiple records to be entered into each of the description items of the transmission/reception format.

According to an inter-organization document information sharing system of claim 6, in the inter-organization document information sharing system of claim 5, the description item multiple entry means includes date/time-provided description item multiple entry means configured to simultaneously enter, into each of the description items, a record of date/time when a document in which the description item is recorded was created.

According to an inter-organization document information sharing system of claim 7, in the inter-organization document information sharing system of claim 6, the date/time-provided description item multiple entry means includes date/time-provided description item multiple recording means configured to create date/time-provided documents in a specified reception-side document category on the basis of a reception-side map and to record the information entered into the date/time-provided description items in corresponding description items in the date/time-provided documents.

According to an inter-organization document information sharing system of claim 8, in the inter-organization document information sharing system of any one of claims 1 to 7, the transmission/reception format definition means includes description item format definition means configured to be able to set a format on a description item.

According to an inter-organization document information sharing system of claim 9, in the inter-organization document information sharing system of any one of claims 3 to 8, the transmission description item entry means includes transmission description item format conversion means configured to convert information in a description item extracted from a transmission-side electronic health record into a format defined in the description item.

According to an inter-organization document information sharing system of claim 10, in the inter-organization document information sharing system of any one of claims 4 to 9, the reception description item recording means includes reception description item format conversion means configured to receive information in a description item and to convert the information into a format defined in the description item of a reception-side electronic health record.

According to an inter-organization document information sharing system of claim 11, in the inter-organization document information sharing system of any one of claims 1 to 10, the transmission/reception format entry/transmission means includes transmission/reception format transmission means configured to (i) directly transmit the transmission/reception format into which the information has been entered to a reception-side electronic health record or (ii) temporarily transmit the transmission/reception format into which the information has been entered to a relay server and transmit the transmission/reception format from the relay server to a reception-side electronic health record.

### Advantageous Effects of Invention

The inter-organization document information sharing system according to claim 1 includes the transmission/reception format definition means and thus defines a versatile transmission/reception format.

The inter-organization document information sharing system of claim 1 includes the transmission/reception format entry/transmission means and thus extracts information from description items in document categories of transmission-side electronic health records, enters the information into the transmission/reception format, and transmits the transmission/reception format.

The inter-organization document information sharing system of claim 1 includes the transmission/reception format reception/recording means and thus receives the transmission/reception format and records the information entered into the transmission/reception format in corresponding description items in document categories of reception-side electronic health records.

Thus, information is shared versatilely regardless of the differences in document category or description item layout between the electronic health records.

In the inter-organization document information sharing system of claim 1, the transmission/reception format definition means is a list of tags indicating description items that need to be shared, that is, has a simple configuration. Thus, the list can be added without affecting the existing operation.

The inter-organization document information sharing system of claim 3 includes the transmission-side map definition means configured to define correspondences between the list of tags indicating the description items that need to be shared and the corresponding description items in the document categories of the transmission-side electronic health records and the transmission description item entry means configured to enter information in the corresponding description items in the document categories into the list of tags indicating the description items that need to be shared on the basis of the transmission-side map definition means. Thus, the configurations of the document categories and description items in the transmission-side electronic health records are temporarily decomposed and converted into versatile expressions.

The inter-organization document information sharing system of claim 4 includes the reception-side map definition means configured to define correspondences between the list of tags indicating the description items that need to be shared and the corresponding description items in the document categories of the reception-side electronic health records and the reception description item recording means configured to record the information entered into the list of tags indicating the description items that need to be shared in the corresponding description items in the document categories of the reception-side electronic health records on the basis of the reception-side map definition means. Thus, the versatile expressions are re-configured into document categories or description items of the reception-side electronic health records.

The inter-organization document information sharing system of claim 5 includes the description item multiple entry means configured to allow multiple records to be entered into each of the description items of the transmission/reception format. Thus, the multiple pieces of description information are shared using the same description item simultaneously.

The inter-organization document information sharing system of claim 6 includes the date/time-provided description item multiple entry means and thus simultaneously enters, into each of the description items, a record of date/time when a document in which the description item is recorded was created. Thus, the information in the description items is entered in a time-series manner

The inter-organization document information sharing system of claim 7 includes the date/time-provided description item multiple recording means and thus creates date/time-provided documents in a specified reception-side document category on the basis of a reception-side map and records the information entered into the date/time-provided description items in corresponding description items in the date/time-provided documents. Thus, the information in the description items is recorded in the time-series documents.

In the inter-organization document information sharing system of claim 8, the transmission/reception format definition means includes the description item format definition means configured to be able to set a format on a description item. Thus, the information in the description items is described without fluctuation in expression.

The inter-organization document information sharing system of claim 9 includes the transmission description item format conversion means and thus converts information in a description item extracted from a transmission-side electronic health record into a format defined in the description item. Thus, the format of the transmission-side electronic health record is converted into a versatile format.

The inter-organization document information sharing system of claim 10 includes the reception description item format conversion means and thus converts received information in a description item into a format defined in the description item of a reception-side electronic health record. That is, the received information in the description item is converted into the format specific to the reception-side electronic health record.

In the inter-organization document information sharing system of claim 11, the transmission/reception format entry/transmission means includes the transmission/reception format transmission means configured to (i) directly transmit the transmission/reception format into which the information has been entered to a reception-side electronic health record or (ii) temporarily transmit the transmission/reception format into which the information has been entered to a relay server and transmit the transmission/reception format from the relay server to a reception-side electronic health record. Thus, the transmission/reception method can be selected in accordance with the cost or purpose.

### BRIEF DESCRIPTION OF DRAWINGS

FIG. 1 is a schematic drawing of an inter-organization document information sharing system according to the present invention.

FIGS. 2A to 2D show examples of a description item tag setting style defined by transmission/reception format definition means.

FIG. 3 is a schematic drawing of transmission/reception format entry/transmission means that extracts description items required in a transmission/reception format from document categories in electronic health records of the transmission-side medical institution/nursing home on the basis of a transmission-side map defined by transmission-side map definition means and enters the extracted description items into the transmission/reception format.

FIG. 4 is a schematic drawing of transmission/reception format reception/recording means that extracts required description items from the transmission/reception format and records the extracted description items in description items in document categories of the electronic health records of the reception-side medical institution/nursing home on the basis of a reception-side map defined by reception-side map definition means.

FIG. 5 is a drawing showing that pieces of information in date/time-provided description items are entered into a transmission/reception format using date/time-provided description item multiple entry means and that date/time-provided multiple documents (“temperature check boards” FIG. 5) are created as a corresponding document category among the electronic health records of the reception-side medical institution/nursing home and the pieces of information in the date/time-provided description items are recorded in corresponding description items in the date/time-provided documents on the basis of the reception-side map.

FIG. 6 is a drawing showing that the format of a description item in an electronic health record of the transmission-side medical institution/nursing home is converted into a format set in a transmission/reception format and the description item is entered into the transmission/reception format and that the format of the description item is converted into the format of an electronic health record of the reception-side medical institution/nursing home and the description item is recorded in the electronic health record.

FIGS. 7A and 7B show two example methods for transmitting a transmission/reception format created from a transmission-side electronic health record to a reception-side electronic health record(s).

### DESCRIPTION OF EMBODIMENTS

An inter-organization document information sharing system according to the present invention includes a server unit, a database, and a terminal.

The server unit is a known computer unit and includes an arithmetic unit, a main memory, an auxiliary storage, an input unit, an output unit, and a communication unit.

The arithmetic unit, main memory, auxiliary storage, input unit, output unit, and communication unit are connected to each other through a bus interface.

The arithmetic unit includes a known processor capable of executing an instruction set.

The main memory includes a volatile memory, such as RAM, capable of temporarily storing an instruction set. The auxiliary storage includes a non-volatile data storage capable of storing an OS or programs.

The data storage may be, for example, an HDD or SSD.

The input unit is, for example, a keyboard.

The output unit is, for example, a display such as an LCD.

The communication unit includes a network interface connectable to a network.

The server unit has means such as transmission/reception format definition means, transmission/reception format entry/transmission means, transmission/reception format reception/recording means, transmission-side map definition means, transmission description item entry means, reception-side map definition means, reception description item recording means, description item multiple entry means, date/time-provided description item multiple entry means, date/time-provided description item multiple recording means, description item format definition means, transmission description item format conversion means, reception description item format conversion means, and transmission/reception format transmission means.

The processor of the server unit performs these means.

The database according to the present invention may consist of the auxiliary storage of the server unit, or may consist of a different auxiliary storage independent of the server unit.

The database stores information handled by an inter-organization document information sharing system.

As with the server unit, the terminal according to the present invention has the hardware configuration of a known computer.

The server unit, database, and terminal according to the present invention are able to communicate with each other through a network.

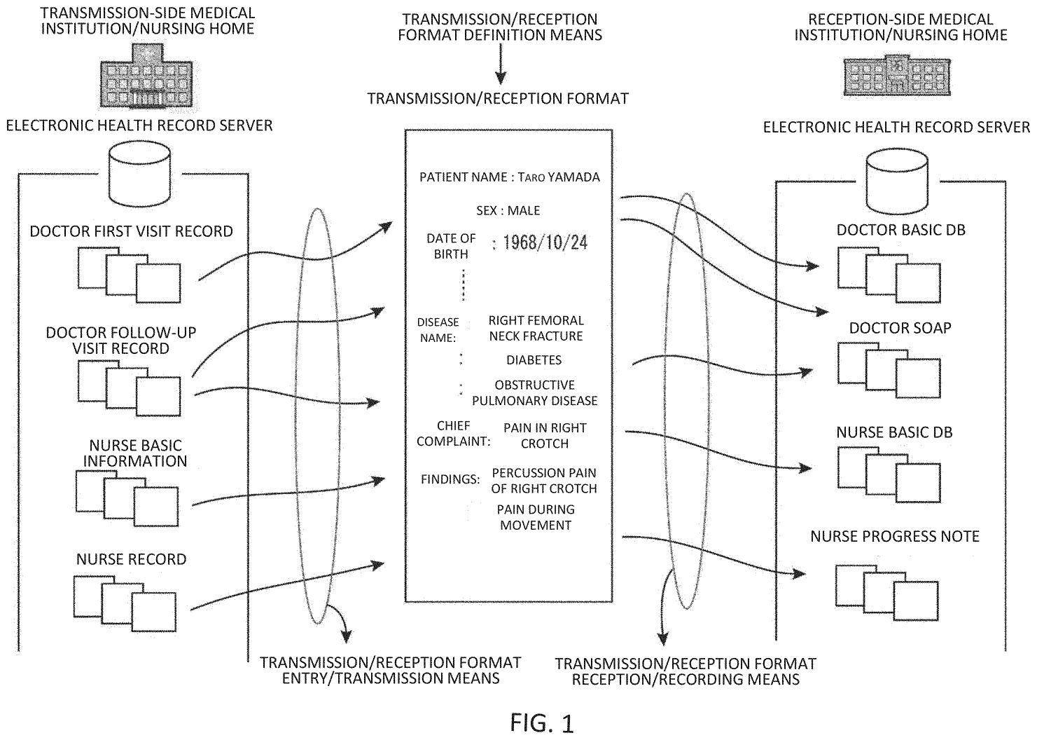

FIG. 1 is a schematic drawing of the inter-organization document information sharing system according to the present invention.

The electronic health record server of a transmission-side medical institution/nursing home includes various document categories such as doctor first visit records. Each document category typically includes multiple description items.

A new document is created by recording description items in any document category at any time, and the creation date/time is recorded in the document.

Information in description items in document categories among transmission-side electronic health records is extracted and entered into corresponding description items in a transmission/reception format defined by the transmission/reception format definition means and then transmitted (transmission/reception format entry/transmission means). The reception-side medical institution/nursing home receives the resulting transmission/reception format and records the information entered in the description items in the transmission/reception format, in corresponding description items in document categories among its electronic health records (transmission/reception format reception/recording means).

Conventionally, document categories and description items in the document categories to be transmitted and received are fixed and converted into a format such as SSMIX-II.

However, this method cannot flexibly cope with document categories among electronic health records or the layout of description items, which vary among medical institutions and nursing homes. The transmission/reception format according to the present invention does not use document categories but rather consist of only description items.

Of course, the description items do not overlap each other, that is, are unique.

Thus, the information in the description items is transmitted and received without being limited by the differences between the configurations of the document categories among the electronic health records.

The reception-side medical institution/nursing home is able to view the received information in the configurations of the document categories and description items of its familiar electronic health records. Also, when necessary, it is able to record the received information in its electronic health records without having to copy and paste them manually.

Note that the number of pieces of information in a description item is not limited to one and multiple pieces of information may be entered in parallel as is shown in “disease name”, “findings:”, and the like in the transmission/reception format of FIG. 1 (description item multiple entry means). The description item multiple entry means will be described later.

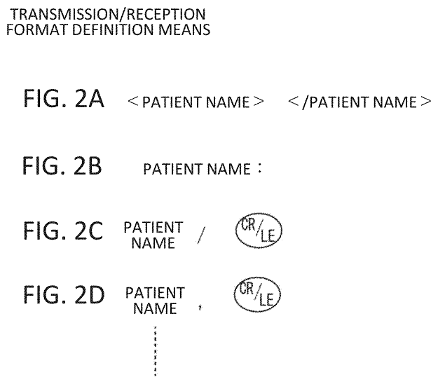

FIGS. 2A to 2D show examples of a description item tag setting style defined by the transmission/reception format definition means.

FIG. 2A shows a setting using XML. Information corresponding to a description item is described between <description item name> and </description item name>.

FIG. 2B shows a JSON format. “:” is placed subsequent to a description item name, and information corresponding to the description item is described subsequent to “:”.

As are seen in “disease name,” “findings,” and the like in the transmission/reception format of FIG. 1, multiple pieces of information may be entered into each description item (description item multiple entry means). Any delimiter may be set between multiple pieces of information.

FIGS. 2C and 2D show a so-called telegram format or a comma-separated values (CSV) format. Subsequent to a description item name, pieces of information are separated by “/” or “,” (inter-information delimiter), and description items are separated by CR/LF or the like (inter-item delimiter).

In the transmission/reception format, a tag list of description items that are not required to share information may be skipped.

That is, only required description items may be entered and recorded.

Various description items are required for each of many hospital departments.

Any number of any tags can be added with respect to those description items without affecting the medical institution or nursing home using the existing list as long as the tags are unique and do not overlap each other. When transmitting or receiving description items, those in which no information is entered may be skipped. Thus, information in various description items of many hospital departments can also be flexibly handled without limitation. Information can be shared smoothly by entering information only into description items that are required to share information (for example, disease name, doctor article, examination results, etc.).

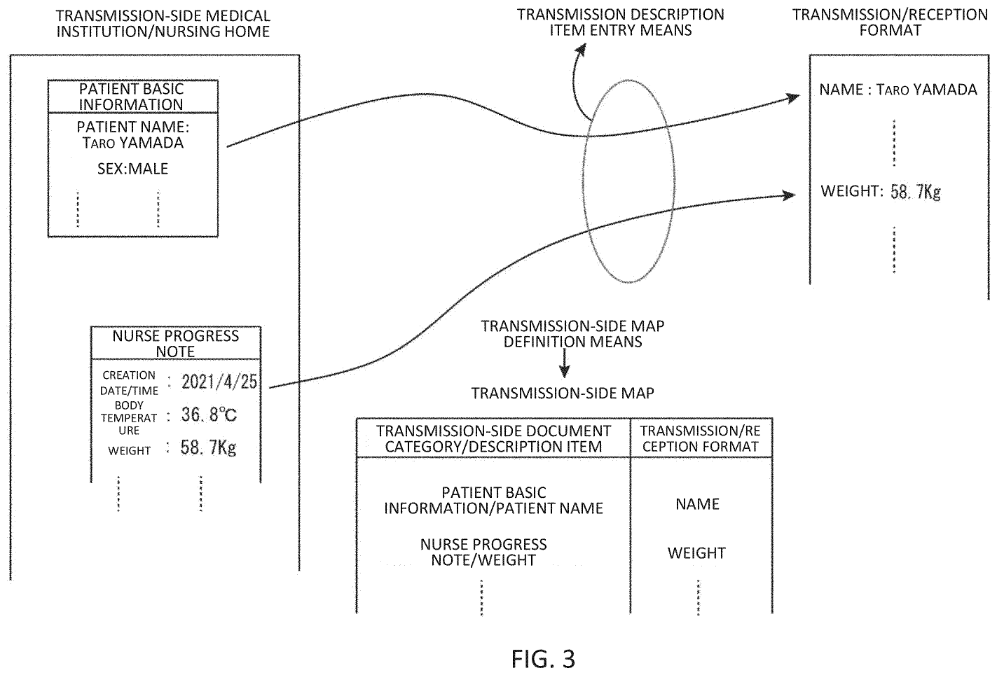

FIG. 3 is a schematic drawing of transmission/reception format entry/transmission means that extracts description items required in the transmission/reception format from document categories among the electronic health records of the transmission-side medical institution/nursing home and enters the extracted description items into the transmission/reception format on the basis of a transmission-side map defined by transmission-side map definition means.

The transmission-side map shows a list of the correspondences between pairs of document categories among the transmission-side electronic health records and description items recorded in the document categories to be entered into the transmission/reception format and corresponding description items in the transmission/reception format.

The latest information in the description items in the document categories specified by the transmission-side map is extracted from the transmission-side electronic health records and entered into the description items in the transmission/reception format specified by the transmission-side map.

Typically, description items such as “weight” are each recorded in multiple document categories such as nursing records and rehabilitation records among the electronic health records in a scattered manner.

However, weight or the like is not necessarily measured for each document category. For example, weight or the like measured and recorded in a nursing record by a nurse is often transcribed in records falling under different job categories.

In such a case, only the information in the transcription source, which is the latest record, may be extracted and entered into the transmission/reception format.

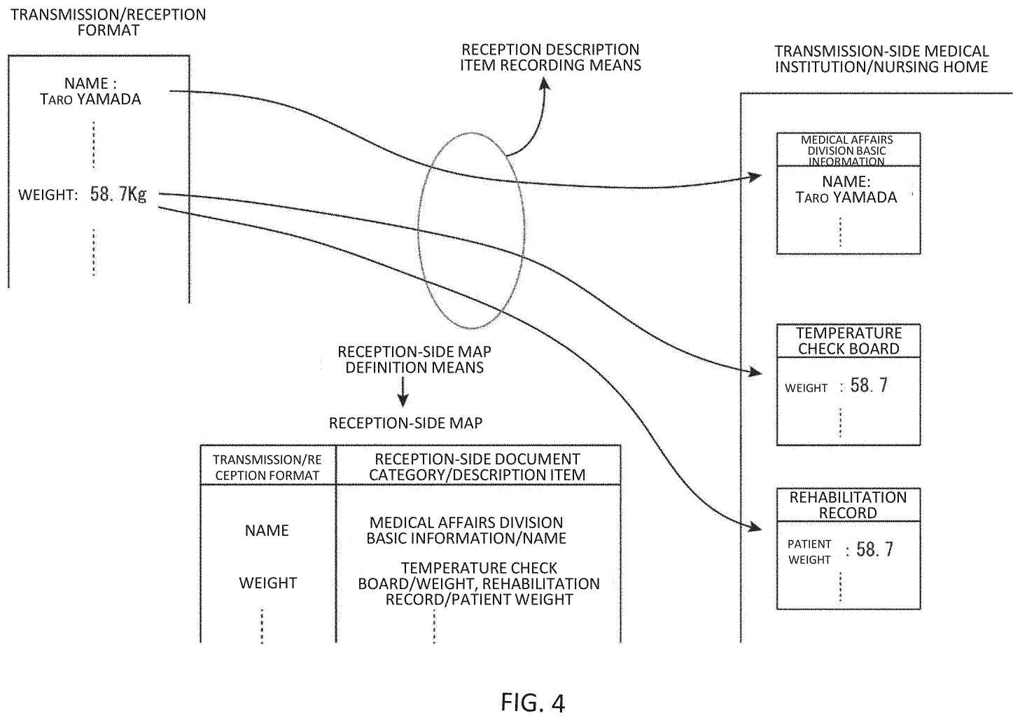

FIG. 4 is a schematic drawing of transmission/reception format reception/recording means that extracts the required description items from the transmission/reception format and records the extracted description items in corresponding description items in document categories among the electronic health records of the reception-side medical institution/nursing home on the basis of a reception-side map defined by reception-side map definition means.

The reception-side map shows a list of the correspondences between the description items in the transmission/reception format and pairs of the document categories among the reception-side electronic health records and the description items to be recorded in the document categories. The information in the description items is extracted from the transmission/reception format and recorded in the corresponding description items in the document categories among the reception-side electronic health records on the basis of this reception-side map.

Typically, description items such as “weight” are each recorded in multiple document categories such as nursing records and rehabilitation records among the electronic health records in a scattered manner. However, weight or the like is not necessarily measured for each document category. For example, weight or the like measured and recorded in a nursing record by a nurse is often transcribed in records falling under different job categories.

In such a case, the information in the description items in the transmission/reception format, which is the latest records, may be recorded in the corresponding description items of the document categories in the reception-side electronic health records on the basis of the reception-side map.

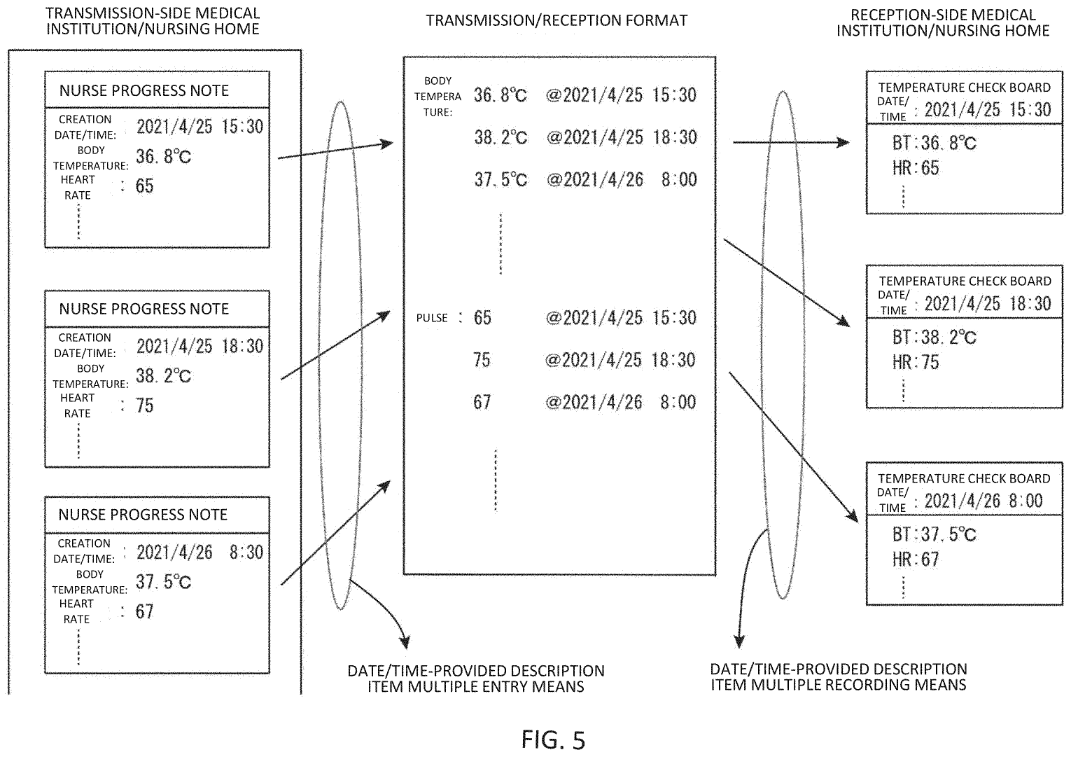

FIG. 5 is a drawing showing that pieces of information in date/time-provided description items are entered into a transmission/reception format using date/time-provided description item multiple entry means (date/time-provided description item multiple entry means) and that multiple date/time-provided documents (“temperature check boards” FIG. 5) are created as a corresponding document category among the electronic health records of the reception-side medical institution/nursing home and the pieces of information in the date/time-provided description items are recorded in corresponding description items (“BT:”, “HR:” in FIG. 5) in the date/time-provided documents on the basis of the reception-side map.

When sharing information between medical institutions and/or nursing homes, it is often necessary to share time-series document information.

Time-series documents (“nursing progress notes” in FIG. 5) are created as a document category among the transmission-side electronic health records, and the creation dates/times of those documents and information in description items (“body temperature” and “heart rate” in FIG. 5) at the dates/times are recorded in the documents. The multiple pieces of information in the description items in the time-series documents are entered into the transmission/reception format along with the creation dates/times of the documents (date/time-provided description item multiple entry means).

Multiple creation date/time-provided documents (“temperature check boards” in FIG. 5) are created as a corresponding document category among the reception-side electronic health records, and the pieces of information in the description items in the transmission/reception format are recorded in the description items (“BT:” and “HR:” in FIG. 5) in the creation date/time-provided documents (date/time-provided description item multiple recording means).

In this way, the time-series documents among the transmission-side electronic health records are reproduced in the reception-side electronic health records. While, in FIG. 5, the recording-destination document category among the reception-side electronic health records is the single category, that is, “temperature check board” for the convenience of description, information in the description items is distributed to multiple document categories in the reception-side electronic health records in some cases. In those cases, multiple document categories may be created in a time-series manner as necessary so that the information in the description items is recorded in the multiple document categories.

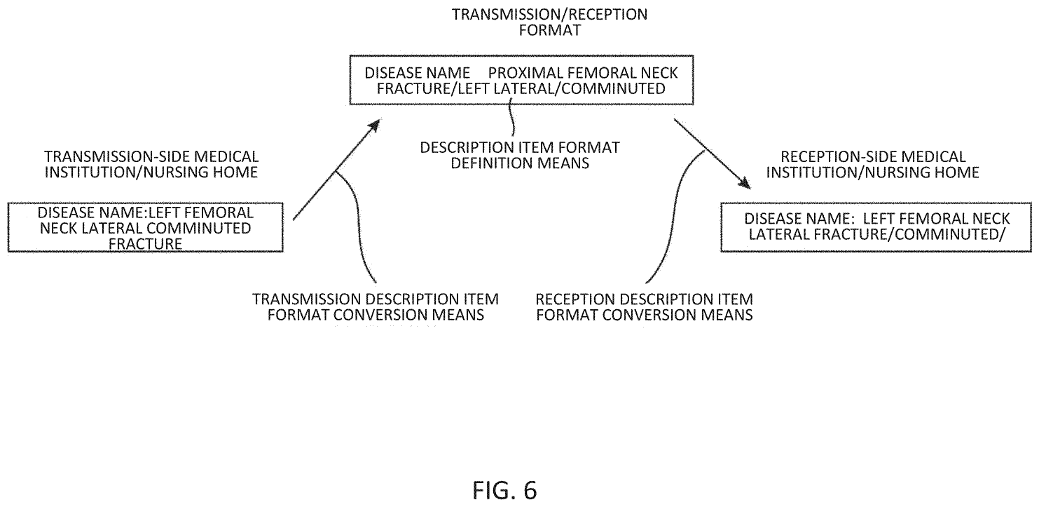

FIG. 6 is a drawing showing that the format of a description item in an electronic health record of the transmission-side medical institution/nursing home is converted into a format (description item format definition means) set in a transmission/reception format and the description item is entered into the transmission/reception format and that the format of the description item is converted into the format of an electronic health record of the reception-side medical institution/nursing home and the description item is recorded in the electronic health record.

A precise format is defined for each description item in many types of electronic health records for convenience in processing or for prevention of fluctuations in description. The above-mentioned failure case of the Saga University occurred due to a lack of consideration of this point.

The format of a description item in a transmission/reception format is previously defined by the transmission/reception format definition means. A corresponding description item in a transmission-side electronic health record is converted into the format of the description item in the transmission/reception format and entered into the transmission/reception format (transmission description item format conversion means).

Then, the description item in the transmission/reception format is converted into the format of a corresponding description item in a reception-side electronic health record and recorded in the electronic health record (reception description item format conversion means).

Thus, the information is shared without having to become conscious of the difference in format between the description items in the electronic health records.

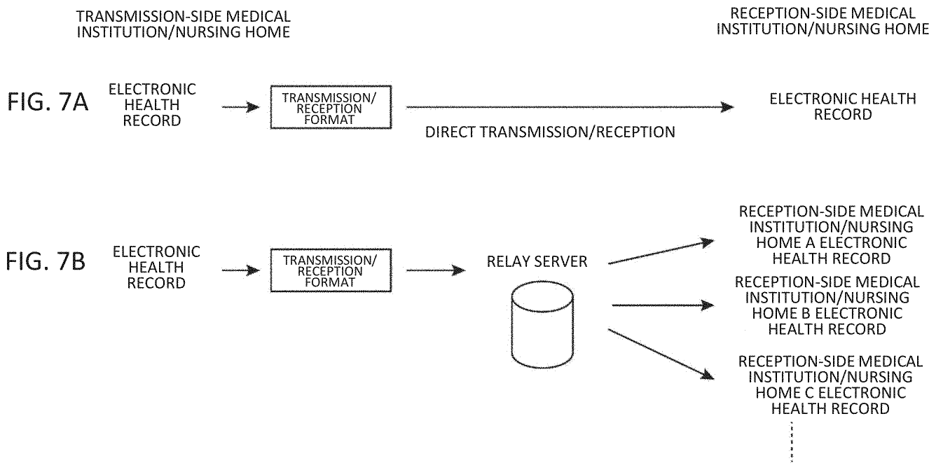

FIGS. 7A and 7B show two example methods for transmitting a transmission/reception format created from a transmission-side electronic health record to a reception-side electronic health record.

The method of FIG. 7A includes creating a transmission/reception format from an electronic health record of the transmission-side medical institution/nursing home and transmitting it to an electronic health record of the reception-side medical institution/nursing home one-to-one using a method such as web services. This method is simple and clear and only requires a minimum cost.

On the other hand, the method of FIG. 7B includes temporarily transmitting a created transmission/reception format to a relay server and distributing it from the relay server to multiple medical institutions and/or nursing homes.

The method of FIG. 7B would be suitable to accomplish an objective as seen in the example of the Saga University. On the other hand, the one-to-one method of FIG. 7A is difficult to manage. While the two examples have been described as the transmission/reception method, these examples are not limiting. Methods such as P2P are also available, and an appropriate method may be selected.

While an embodiment of information sharing between medical institutions and/or nursing homes has been described above, the specific configuration of the present invention is not limited to the embodiment. Design changes and the like in the embodiment are also included in the present invention without departing from the spirit and scope of the invention.

For example, the present invention can also be applied to cases in which document information is shared between organizations other than medical institutions or nursing homes.

Shared document information may be permanently recorded in the electronic health records of the reception-side medical institution/nursing home after the transmission source is described in the shared information. Even if the shared document information is temporarily stored or only viewed, it is useful because the reception-side medical institution/nursing home is able to utilize it in the configuration of the document categories or description items of the facility's familiar electronic health records.

Although not mentioned specifically, it is indispensable to verify the identity of the patient or ensure security against information leakage, as a matter of course.

1. An inter-organization document information sharing system for sharing document information between electronic health records of a plurality of organizations between which configurations of document categories or description items in the document categories do not necessarily match each other, the system comprising:
transmission/reception format definition means configured to define a versatile transmission/reception format;
transmission/reception format entry/transmission means configured to extract information from description items in document categories of transmission-side electronic health records, to enter the information into the transmission/reception format, and to transmit the transmission/reception format; and
transmission/reception format reception/recording means configured to receive the transmission/reception format and to record the information entered into the transmission/reception format in corresponding description items in document categories of reception-side electronic health records.

2. The inter-organization document information sharing system of claim 1, wherein the transmission/reception format definition means defines a list of tags indicating description items that need to be shared.

3. The inter-organization document information sharing system of claim 1, wherein
the transmission/reception format entry/transmission means comprises:
transmission-side map definition means configured to define correspondences between the list of tags indicating the description items that need to be shared and the corresponding description items in the document categories of the transmission-side electronic health records; and
transmission description item entry means configured to enter information in the corresponding description items in the document categories into the list of tags indicating the description items that need to be shared on the basis of the transmission-side map definition means.

4. The inter-organization document information sharing system of claim 1, wherein
the transmission/reception format reception/recording means comprises:
reception-side map definition means configured to define correspondences between the list of tags indicating the description items that need to be shared and the corresponding description items in the document categories of the reception-side electronic health records; and
reception description item recording means configured to record the information entered into the list of tags indicating the description items that need to be shared in the corresponding description items in the document categories of the reception-side electronic health records on the basis of the reception-side map definition means.

5. The inter-organization document information sharing system of claim 1, comprising description item multiple entry means configured to allow a plurality of records to be entered into each of the description items of the transmission/reception format.

6. The inter-organization document information sharing system of claim 5, wherein the description item multiple entry means comprises date/time-provided description item multiple entry means configured to simultaneously enter, into each of the description items, a record of date/time when a document in which the description item is recorded was created.

7. The inter-organization document information sharing system of claim 6, wherein the date/time-provided description item multiple entry means comprises date/time-provided description item multiple recording means configured to create date/time-provided documents in a specified reception-side document category on the basis of a reception-side map and to record the information entered into the date/time-provided description items in corresponding description items in the date/time-provided documents.

8. The inter-organization document information sharing system of claim 1, wherein the transmission/reception format definition means comprises description item format definition means configured to be able to set a format on a description item.

9. The inter-organization document information sharing system of claim 3, wherein the transmission description item entry means comprises transmission description item format conversion means configured to convert information in a description item extracted from a transmission-side electronic health record into a format defined in the description item.

10. The inter-organization document information sharing system of claim 4, wherein the reception description item recording means comprises reception description item format conversion means configured to receive information in a description item and to convert the information into a format defined in the description item of a reception-side electronic health record.

11. The inter-organization document information sharing system of claim 1, wherein the transmission/reception format entry/transmission means comprises transmission/reception format transmission means configured to (i) directly transmit the transmission/reception format into which the information has been entered to a reception-side electronic health record or (ii) temporarily transmit the transmission/reception format into which the information has been entered to a relay server and transmit the transmission/reception format from the relay server to a reception-side electronic health record.
